(19) United States
(12) Patent Application Publication
(10) Pub. No.: US 2010/0016957 A1
(43) Pub. Date: Jan. 21, 2010

(54) DEVICE AND METHOD FOR CONTROLLED DELIVERY OF CHEMICAL SUBSTANCES

(76) Inventors: Edwin Jager, Linkoping (SE); Daniel Carlsson, Stockholm (SE); Mia Skoglund, Linkoping (SE); Magnus Krogh, Linkoping (SE); Anders Selbing, Linkoping (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/302,470

(22) PCT Filed: May 28, 2007
(86) PCT No.: PCT/EP07/04715

§ 371 Date: Aug. 6, 2009

Related U.S. Application Data
(60) Provisional application No. 60/808,549, filed on May 26, 2006.

(30) Foreign Application Priority Data
Sep. 14, 2006 (SE) 0601902-0

(51) Int. Cl. A61F 2/06

(52) U.S. Cl. 623/1.42

(57) ABSTRACT

A medical device for introduction into a body comprises a body-insertable part (1, 3, 7, 10, 20, 30, 35, 40, 60, 70, 80) having a first electroactive polymer (13), integrated with the body-insertable part and comprising an electrically controllably releasable first substance. There is further disclosed a method for delivering a chemically active substance into a body.

### TECHNICAL FIELD

The present disclosure relates to a device and method for controlled delivery of chemical substances. More specifically, the disclosure relates to a device and method for delivery of drugs to precise locations inside a body.

### BACKGROUND

The present disclosure addresses the problem of delivering chemical substances to precise locations on demand. Such substances may be reagents or pharmacologically active substances which are contained in a polymer layer covering the complete or a partial surface of a medical device or surgical instrument.

Drug delivery devices are available in many forms, ranging from pharmaceutical preparation methods (pills, tablets, mixtures etc.) to controlled release (electricity, mechanical, variable solubility etc.) and facilitated transport (iontophoresis, aerosol).

Applications can also be classified according to the location where the substance is to be applied, like surface applications (cutaneous, rectal, vaginal, nasal, oral, airways), injection or infusion (subcutaneous, intramuscular, intravenous, intraspinal, extradural, intrathecal, intravascular).

One example of a recent development are drug coated intravascular stents (examples: EP0822788, WO2003026713, EP1117351, U.S. Pat. No. 5,545,208) which can be categorised as intravascular application devices with solubility release of active pharmacological substances. The intravascular introduction of a drug-containing device starts the drug-dissolving process and the drug will spread into the surroundings by diffusion. When said drug-dissolving process is proceeding, much of the drug will be locally diluted in and transported away from the target area by the blood stream. The drug will therefore have to be administered in much higher concentration to exert its function and only a fraction of the given substance will reach the aimed target, while most of the drug will affect areas not intended to be treated.

Conjugated or conductive polymers, such as polypyrrole, can be electrochemically oxidised and reduced. This oxidation and reduction is accompanied with the transport of ions and solvents into and out of the conductive polymer. This redox reaction changes the properties of polypyrrole such as the conductivity, colour, and volume. The volume change can, for instance, be used to build actuators (See Q. Pei and O. Inganäs, “Conjugated polymers and the bending cantilever method: electrochemical muscles and smart devices”, Advanced materials, 1992, 4(4), p. 277-278. and Jager et al.,” Microfabricating Conjugated Polymer Actuators”, Science 2000 290: 1540-1545).

Also, these materials may be used for the release of active species such as drugs. Usually, the transported ions are small ions such as Na+ and ClO4-. However, these can be exchanged for active species such as drugs.

U.S. Pat. No. 6,394,997 discloses the use of electroresponsive copolymer gels that are encapsulated for drug delivery applications. When the gel is actuated by an externally applied field such as electrical current, electric field etc., at least a portion of the wall of the container or enclosure is deformed to perform a mechanical function. The drug delivery system disclosed consists of a permeable membrane which encloses a layer of expandable copolymer gel. Inside the layer of expandable copolymer gel is a thin, flexible sack, which contains a dose of a drug. Two electrode means are also positioned in the device, of which at least one is attached to the gel. On operation an electric potential between the electrodes is applied for a sufficient time to rupture the sack. As the expandable copolymer gel continues to contract, the drug escapes from the ruptured sack and is forced into the body through the permeable membrane.

A similar pump concept, where electroactive polymers are employed to “press out” drugs from a contractable or expandable enclosure is disclosed in US20040068224 and US20040182704.

The use of polymers with charged redox sites, such as polypyrrole, for controlled delivery of ionic bioactive chemicals was disclosed by Miller et al, U.S. Pat. No. 4,585,652.

A variant/improvement is disclosed in WO0213785. Here a non-faradaic release profile, defined as a burst, is described. Also, in WO0213784 it is noted that spontaneous release of the active molecules by ion exchange was a problem for controlled drug release electrodes based on electroactive polymers. Building a bilayer structure by adding a second polymer layer on top of the electroactive polymer, slows or even stops the spontaneous release. The use of electroactive polymers in release pads is disclosed in WO0125406.

Instead of using the redox properties of conducting polymers directly, WO9833552 discloses a different, indirect mechanism for electrorelease. Generation of protons by electrochemical oxidation at a second functional group, such as a cysteine group, causes breakage of the ionic bond that binds the charged species to the matrix, thereby releasing the electoreleasable species.

Electropolymer coated microelectrodes, smaller than 50 μm, for sensor applications are disclosed in WO9002829. The coated microelectrodes can also be used for controlled release.

U.S. Pat. No. 6,049,733 discloses incorporating ion exchange materials, (polypyrrole is included in their definition of ion exchange materials) in a drug reservoir of an electrotransport system for transdermal drug delivery. The ion exchange material is used to immobilize competitive ions that are generated in the electrotransport process and that compete with the drug to be delivered through the skin. The ion exchange material is not used as the drug reservoir, only as a means to improve the drug delivering properties of the transdermal drug delivery system.

Several implantable medical devices with polymeric coatings and passive diffusion of the agents can be found. For instance, thin polymeric coatings of implantable devices with metallic surfaces, such as stents, for protection and biocompatibility are described in WO0139813. The polymeric coating is prepared by electropolymerisation of oxidisable monomer, including polypyrrole derivatives complexed with anionic molecules. Bioactive or bioreactive agents are incorporated in the polymer film, preferably covalently bound. They can even be electrostatically complexed. These agents are released in a predictable way over time as a function of the degradation of the conjugation bond. However, all of these devices will begin to release the agent immediately upon introduction into the body.

A similar device is disclosed in U.S. Pat. No. 6,468,304. This patent describes electrochemical coating of implants such as stents with conductive polymers that are sequentially loaded with bioactive, charged molecules by oxidation or reduction of the conductive polymer. The coating increases the biocompatibility and the biomolecules are slowly released (passive release) during the implantation, ensuring prolonged effect of the active substances.

Other implanted devices with polymer coatings are disclosed in U.S. Pat. No. 6,309,380.

U.S. Pat. No. 6,326,017 describes a localized delivery of agents to blood vessels using polymer layers and diffusion of these agents from the polymer layers. Electrically induced or in other ways actively induced agent delivery is not described.

In U.S. Pat. No. 5,674,192 a catheter with a PTCA balloon that has a hydrogel layer is presented. The hydrogel is filled with a biological agent such as nucleic acids before the procedure. Upon expansion of the balloon at the site of interest in a blood vessel the biological agent is pressed out of the hydrogel layer due to the expansion force of the balloon on the vessel.

Several examples of injection of drugs into the blood vessel using double walled balloon catheter have been presented (see for instance U.S. Pat. No. 4,994,033 and U.S. Pat. No. 6,149,641). The outer balloon layer has a means (e.g. porous pores or holes) of allowing drugs to be applied through the catheter shaft into the blood vessel.

There is a need for a device that is capable of locally delivering, momentarily and on demand, substances at a specific anatomical location. Such devices would be most valuable to increase the effectiveness of treatment and to decrease the risk for side-effects.

### SUMMARY

A general object of the present disclosure is to provide a device and method, which overcome the disadvantages of the prior art.

Another object of the present disclosure is to provide an improved device and method for controlled delivery of chemical substances, in particular for pharmacologically active substances.

The objects are wholly or partially achieved by devices and methods according to the appended independent claims. Embodiments are set forth in the dependent claims and in the following description and drawings.

According to a first aspect, there is provided a medical device for introduction into a body. The device comprises a body-insertable part having a first electroactive polymer, integrated with the device and comprising an electrically controllably releasable first substance.

By “electrically controllable”, is meant that the release of the first substance can be effectively increased or decreased by applying, removing or varying an electrical signal to the electroactive polymer.

Such a device enables release of the first substance at a desired position within the body and at a desired point in time. The device may also be used as a medical device, e.g. in applications outside the body, e.g. in transdermal applications.

The first substance may be a biologically active substance.

Alternatively, the first substance may be a precursor or prodrug to a biologically active substance.

The body-insertable part may also comprises a second electroactive polymer, integrated with the body-insertable part and comprising an electrically controllably releasable second substance.

The second substance may be a biologically active substance.

Alternatively, the second substance may be a precursor or prodrug to a biologically active substance.

The second substance may be a component, which when reacting with first substance forms a biologically active substance.

The second substance may be a catalyst or initiator for interaction with the first substance for forming a biologically active substance.

The skilled person realizes that two or more such EAP portions may be provided, and hence the body-insertable part may include e.g. two, three, four, five, six, etc. different, and optionally individually controllable, portions for releasing a respective substance, analogously with what has been described above. With multiple EAP portions, these may contain the same substance, or different substances.

The device may further comprise means for controlling a mechanical movement of the body-insertable part, such as a gripping function or a shape.

Such means may include, but is not limited to, an EAP actuator, a shape memory alloy actuator, or any type of mechanic or micro-mechanic actuator. The movement controlling means may be arranged to e.g. bend, expand, contract, rotate, translate, etc. the body-insertable part, or a portion thereof. The movement may be performed with a view to positioning the electroactive polymer, e.g. so as to press it against a tissue portion to which the substance is to be delivered.

The movement controlling means may comprise an actuator. In one embodiment, the actuator comprises a third electroactive polymer. The movement controlling means may be electrically controllable.

The movement controlling means and the first electroactive polymer, and the second electroactive polymer, if any, may be individually controllable.

The device may further comprise a control device for providing at least one control signal to at least one of the first electroactive polymer, the second electroactive polymer, if any, and the movement controlling means, if any.

The control signal may be an electrical signal for controlling release or movement, respectively.

The control device may comprise means for providing at least two control signals with a time delay therebetween. The time delay may be programmable.

The first electroactive polymer, or the second electroactive polymer, if any, may be arranged at a portion of the body-insertable part, which is to contact a predetermined portion of the body.

The first electroactive polymer, the second electroactive polymer, if any, or the third electroactive polymer, if any, may be arranged at an outer portion of the body-insertable part, preferably on an outer surface of the body-insertable part.

The first electroactive polymer, the second electroactive polymer, if any, or the third electroactive polymer, if any, may be arranged at an inner portion of the body-insertable part, preferably on an inner surface of the body-insertable part.

The first electroactive polymer, and at least one of the second electroactive polymer and the movement controlling means, may be arranged on the same side of body-insertable part.

As another option, or complement, the first electroactive polymer, and at least one of the second electroactive polymer and the movement controlling means, may be arranged as layers, e.g. on top of each other.

The first electroactive polymer, and at least one of the second electroactive polymer and the movement controlling means, may be arranged on opposite sides of the body-insertable part.

The body-insertable part may comprise a medical device, such as a catheter, a needle, a guidewire, a stent, a balloon, an anchoring device, an aneurysm coil, etc.

Alternatively, the body-insertable part may comprise a surgical tool, such as a knife, scissors, clamp, forceps, etc.

The body-insertable part may comprise a tool for microsurgery.

The body-insertable part may also comprise a liner for a body lumen, wherein the first electroactive polymer is on an outer side of the liner.

A body lumen may be any substantially tubular body structure, such as a blood vessel, an intestine, lymphatic vessel etc.

The body-insertable part may comprise a substantially tubular structure.

The body-insertable part may comprise a substantially spiral-shaped or helical structure.

The body-insertable part may comprises a plurality of foldable flaps.

At least one of the flaps may meet the carrier at an angle between 0 and 90 degrees.

The body-insertable part may comprise a filter device. In such a device, the first electroactive polymer may be arranged on a filter member.

The body-insertable part may comprise a neural connector. In such a device, the first electroactive polymer may be on a nerve-facing side of the body-insertable part.

The body-insertable part may further comprise a carrier device. Such a carrier device may have the form of a needle, a catheter, a guidewire etc.

The first electroative polymer may be formed as a separate part, which is mounted on the body-insertable part.

Alternatively, the first electroactive polymer may be formed directly on the body-insertable part.

The first substance may be selected from a group consisting of steroids, growth factors, resodilatives, antiproliferatives, antibiotics, cytostatics, cytotoxics, immuno-suppressives, anti-inflammatories, thrombolytics, anti-thrombolytics, pro-coagulatives, anti-coagulatives, vaso-delatives, neuro-transmitters and neuro-modulators.

Other substances are not excluded

The electroactive polymer may be a conducting polymer selected from a group consisting of pyrrole, aniline, thiophene, para-phenylene, vinylene and phenylene polymers and copolymers thereof, including substituted forms of the different monomers.

Other conducting polymers having similar properties are not excluded According to a second aspect, there is provided use of a device as claimed in any one of the preceding claims for vascular surgery, microsurgery, brain surgery, coronary surgery, treatment of emboli (stroke), treatment of aneurysm.

According to a third aspect, there is provided a method for delivering a chemically active substance into a body. The method comprises introducing into the body a body-insertable part comprising a first electroactive polymer integrated with the body-insertable part and comprising an electrically controllably releasable biologically active substance, and delivering the chemically active substance by providing an electrical signal to control the first electroactive polymer.

The method may be performed in vivo. Alternatively, the method may be performed on non-living tissue.

According to a fourth aspect, there is provided a method for delivering a chemically active substance into an in vitro system. The method comprises introducing into the system a body-insertable part comprising a first electroactive polymer integrated with the body-insertable part and comprising an electrically controllably releasable biologically active substance, and delivering the chemically active substance by providing an electrical signal to control the first electroactive polymer.

The method may further comprise providing a second control signal for controlling a mechanical movement of the body-insertable part, such as a gripping function or a shape.

A device and method as described above have several advantages. When used for surgery, they reduce time, as a second drug delivery tool does not have to be inserted. This means that a new type of procedure, not previously possible, is rendered possible. Substances can thus be released at the site of interest, precisely where the operation is performed. The amount of substance can also be precisely controlled, and the dose can be given with exact timing in relation to the progress of the procedure. Several types of drugs (individually triggered) can also be integrated on one tool and be actively delivered simultaneously or sequentially.

Yet another advantage is that the EAP layers, such as PPy, may be relatively thin (typically 1-100 μm), which means that the addition of such layers does not substantially increase the size of the medical device.

Devices for medical purposes are described in publication WO 00/78222. Such mechanical devices may be catheters and catheter systems, as well as devices positioned by means of catheters, like clamps, forcepses, expandable tubes, constricting tubes and devices having other geometrical forms not yet known.

Integration of a microsurgical tool and an electroactive drug delivery layer means new possibilities to pharmacologically administer local treatment with minimal affect on adjacent and distant tissues.

However, the use of the devices and methods described herein is not limited to insertion in human or animal bodies, but can also be used in in vitro biomedical systems. Thus, the integration of EAP portions that incorporate chemical substances in micro-tools placed in, or entered in, for instance channels, holes, or cavities in microfluidic chips may be used to deliver, on demand, chemical substances both as single release, repeated releases but also sequential release of different substances. These properties can be used for delivering reagents in a chemical test system, pharmacological substances in living cell tests or, in drug screening test systems.

### BRIEF DESCRIPTION OF THE DRAWINGS

FIGS. 1a-1c schematically illustrate examples of parts of medical devices or surgical tools.

FIGS. 2a and 2b schematically illustrates a medical device for introduction into a body lumen.

FIGS. 3a-3e schematically illustrates a device for delivering the chemical substance to the inside of a body lumen.

FIG. 4 schematically illustrates sectional view of a liner structure, which may be used in the embodiments described herein.

FIGS. 5a-5b schematically illustrate further liner structures, which may be used in the embodiments described herein.

FIGS. 6a-6d schematically illustrate further devices for delivering the chemical substance to the inside of a body lumen.

FIG. 7 schematically illustrates a medical device introduced into a cannula or catheter.

FIG. 8 schematically illustrates an embolic capture device.

FIGS. 9a-9d schematically illustrate a nerve connector.

FIG. 10 schematically illustrates a system, comprising a medical device 1 having a body-insertable part 2 and a control device 90.

### DESCRIPTION OF EMBODIMENTS

Initially, this description will focus, by way of example, on a method for providing an electroactive polymer portion, in the form of a layer comprising a controllably releasable chemical substance. Subsequently, some examples of devices, on which such layers may be provided, will be described.

In a first example polypyrrole was electrochemically synthesised from water based electrolytes, containing pyrrole monomers and different pH indicators, such as phenol red, bromcresol green, and bromthymol blue, on surfaces such as metal wires, gold coated plastic substrates and glass wafers with a patterned gold layer. [andse1]During synthesis these anionic pH indicators were incorporated in the polypyrrole film. To release the pH indicator molecules, the samples were submerged in a salt solution, such as 0.15M NaCl or 0.1M sodium dodecylbenzenesulphonate, and a low potential was applied (typically −1V vs Ag/AgCl). Within a few seconds after the potential had been applied, the electrolyte around the polypyrrole became coloured, indicating that pH indicator had been released. Different colours for different pH indicators and different pH of the electrolyte were demonstrated. By oxidizing the polypyrrole, the release would cease. Further release could be initiated again, by reapplying a negative potential hence reducing the polypyrrole, until the pH indicator in the polypyrrole film had been depleted.

The skilled person will understand that this is merely a demonstration of the principle and that the present disclosure is not limited to these moieties. The skilled person realizes that any (positively or negatively) charged substances can be released, as for instance disclosed in WO0213785.

In a second example, dexamethasone phosphate, a common drug used for treatment of inflammation, has been incorporated in polypyrrole during synthesis in a similar way as the pH indicators in Example 1 above. Polypyrrole was synthesized from a water based electrolyte containing 0.1 M pyrrole and 2 mM dexamethasone phosphate disodium (DMP) using galvanostatic polymerization. Acid form of DMP is more stable than regular DMP, and cation exchange was therefore carried out prior to electropolymerization. The polymerisation current density was ˜0.1 mA/cm2 and polymerization time 2 hours. The resulting polymer film was activated in 0.15M NaCl by applying a potential sweep from 0V to −1 V and back to 0V at a speed of 5 mV/s. This was repeated three times and resulted in the release of dexamethasone phosphate into the solution.

It is also possible to first form the EAP portion, and thereafter provide the chemical substance.

The description will now focus on the devices upon which the chemical substance containing electroactive polymer layer may be applied.

The drug delivery layer can be integrated into e.g. a medical device or surgical tool, which may result in new possibilities, and new or improved treatments.

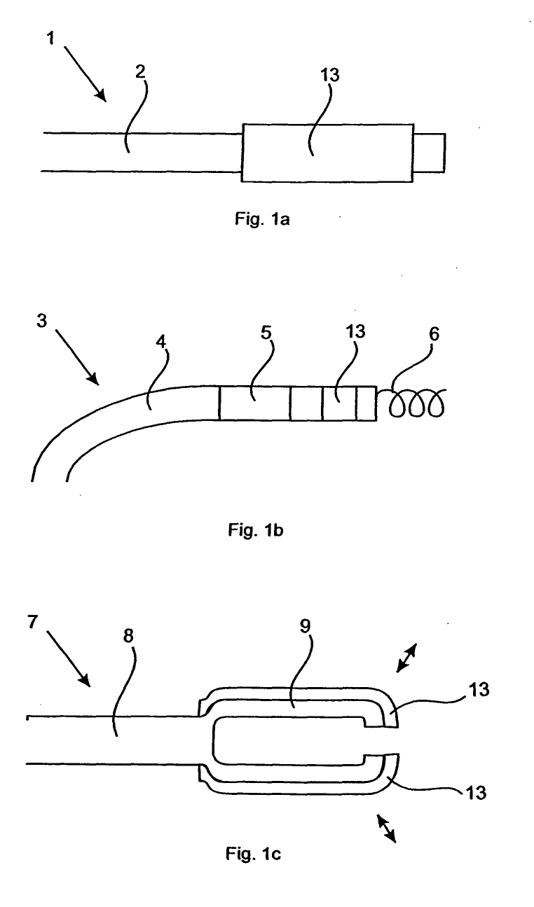

FIG. 1a illustrates a section 2 of a medical device 1 for insertion into the body, such as a catheter. The catheter further comprises an electroactive drug delivery portion 13.

The electroactive polymer drug delivery portion 13 in the embodiments disclosed herein may be arranged as a layer, covering or coating on a body-insertable part 2 of the device 1.

FIG. 1b illustrates a section 4 of a cardiac lead 3, for instance as is used for pacemakers or implantable cardioverter defibrillators, in a manner known to those skilled in the art. The lead 3 comprises two electrodes 5 and 6 that are used for the cardiac therapy. The electrode 6 is coil shaped and may be screwed into the heart tissue for anchoring. Other anchoring solutions are known to those skilled in the art. The lead 3 further comprises an electroactive drug delivery portion 13. The portion 13 may, for instance, be used to release medication that will aid the ingrowth of the lead. For example, steroids, anti-coagulants, anti-inflammatory agents or drugs may be used to reduce the foreign body response. It is contemplated that the drug delivery portion 13 may be positioned at any place on the lead 3, for instance (partially) on the electrodes 5 or 6.

FIG. 1c illustrates a section 8 of a dilator 7 comprising EAP drug delivery portions 13 on the outside of the forceps 9. The forceps 9 can be moved by an external means. While using the dilator in a surgical procedure, a drug may be released from the forceps 9 during the procedure, without having to change or remove the dilator.

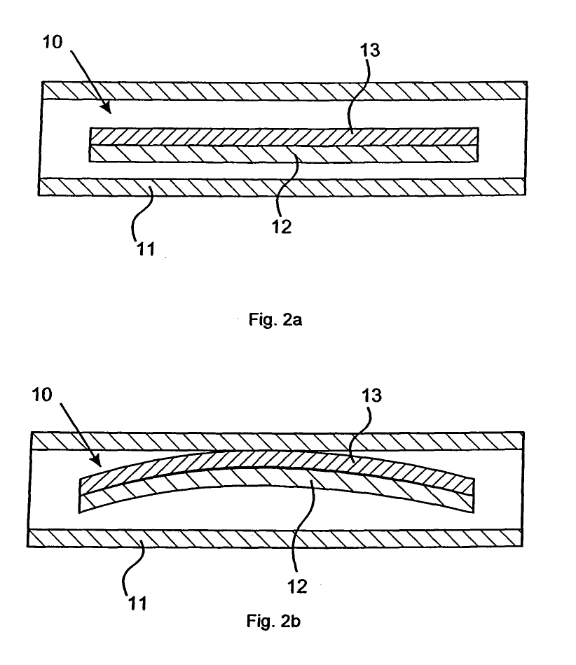

FIG. 2a schematically illustrates a part of a medical device or surgical tool 10 that has been inserted into an area of the body, such as a lumen, for instance a blood vessel. The medical device comprises a part 12 that can be moved and an (EAP) drug delivery portion 13. The medical device, or part thereof, is brought in to contact or proximity of the area to be treated by actuating the movable part 12 (see FIG. 2b). Hereafter the EAP drug delivery portion is actuated and the drugs are released from the portion. The movable part may be a steerable or bendable tip for instance of a guide wire or catheter, an inflation balloon, an embolic coil for treating aneurysm, a filter or basket device, a tubing, a sheath, a scalpel, a marker band or other medical devices that may be actuated by a mechanical means such as spring loaded, wire pulled, twisting, pushing, “expanders”, shape memory effect, bimetal effect, thermal expansion, piezoelectric effect, EAP actuation, (electro-)magnetic, electric, hydrogel, osmotic swelling, or other actuation means as know by those skilled in the art or common in the medical device industry.

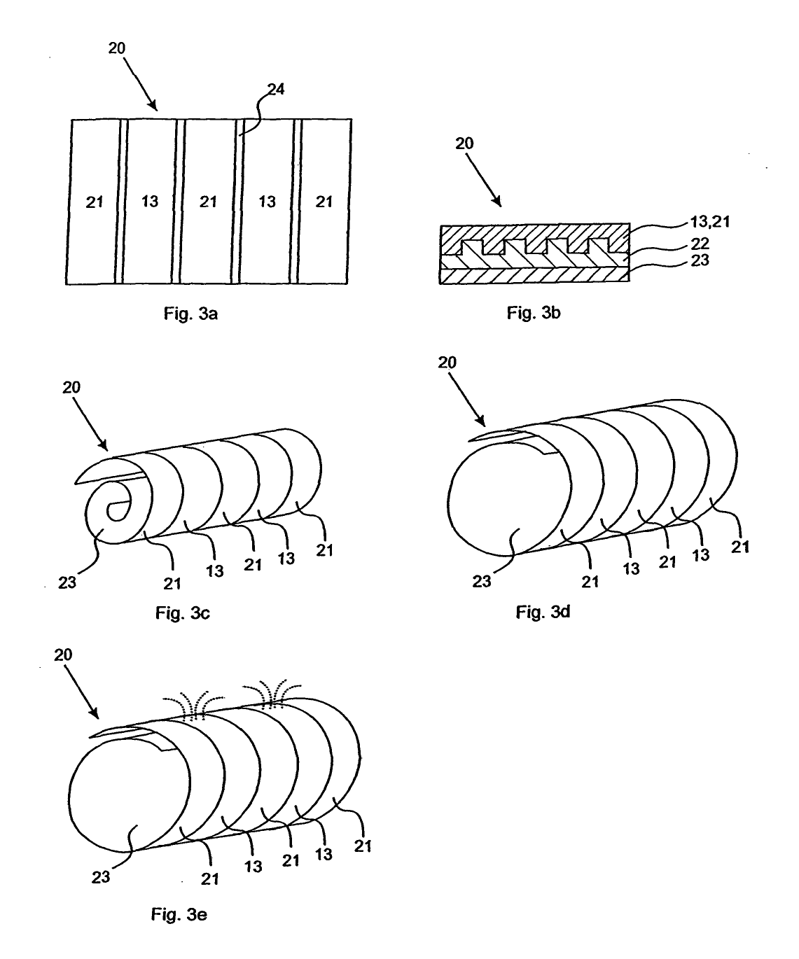

FIGS. 3a-e illustrate a medical device 20 with an integrated (EAP) drug delivery portion 13 that is mechanically activated by an electroactive polymer 21.

FIG. 3a illustrates an embodiment of such a device, a so called liner, which may be introduced in a body lumen or a body fluid channel, such as a blood vessel.

FIG. 3a shows the “top view” of the liner, the part that will be in contact with the lumen surface. The area is divided in sections comprising either an EAP portion that takes care of the mechanical function 21 or an EAP portion that takes care of the drug delivery function 13. These sections 13, 21 may be electrically insulated from each other by a part 24 so that the sections can be individually activated. The insulating part 24 may be a slit or channel separating the individual sections, or it may be an insulating material, for instance a non-conductive polymer.

FIG. 3b shows a cross section of the liner 20 along one of the sections 13 or 21. The liner comprises an EAP portion or layer 13 or 21 that will form the “outside” of the device (facing the lumen surface), and a second non-EAP layer 22 and preferably a third non-EAP layer 23 that will form the “inside” of the device (facing the lumen). The layer 22 may be an electrically conducting layer, for instance gold. The layer 22 may comprise rigid beams or other stiff elements that are perpendicular to the rolling direction of the liner 20. The beams may control the movement as disclosed in WO03039859. The layer 22 may be patterned in order to allow individual control of the layers 13 and 21. The non-EAP layer 23 may be a polymer that insulates the above mentioned conducting layer 22 and/or a blood compatible layer used in order to improve the blood compatibility of the liner.

For more details on the function of such a liner/sheet and the rigid beams for controlling the movement, reference is made to WO03039859 the entire contents of which is hereby incorporated by reference.

The electrical interconnects between the different sections for controlling each section have been omitted from FIGS. 1-8 for clarity. It is known to those skilled in the art, how such interconnects may be designed.

The liner 20 may be used as a connector/liner with drug. Pharmacologically active substances may be incorporated in an EAP layer on a sheet of material suitable for intravascular use and release therefrom.

For instance, Paclitaxel (or derivatives or analogs thereof), a drug used to treat or prevent hypertrophy of the vascular wall in association to PTA and PTCA, may be incorporated in polypyrrole layers on sheets of material, here called liner, suitable for intravascular insertion. The sheet has an electroactive polymer, such as polypyrrole, layer 21 for its mechanical function. The layer will on activation make the sheet 20 roll up to form a tube that can be inserted in a contracted state (FIG. 3c) into a body lumen, such as a blood vessel (omitted from FIGS. 3c-3e). Once inside the lumen, e.g. blood vessel, the sheet can be electrically activated to expand and press against the vessel wall (FIG. 3d). When the liner is in contact with, or in close proximity to, the vessel wall, one or several drugs that are located in other EAP layers on the outside of the liner can be released by electrical stimulation (FIG. 3e), and shortly afterwards a high local concentration of the drug will result. The liner can be a permanent implant, or alternatively, the liner may be contracted and removed from the blood vessel. It is contemplated that the drug A that is released may be a precursor or prodrug to active substance A′, that is formed after release from the EAP layer, for instance by metabolization (e.g. hydrolysis) or enzymatic reaction of the precursor or prodrug.

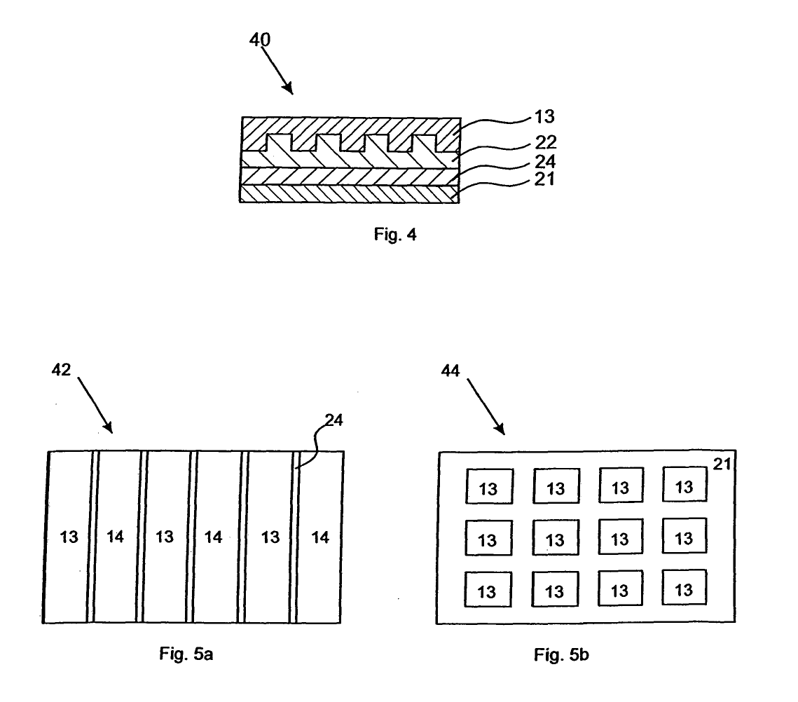

FIG. 4 illustrates an alternative design of the drug delivery liner. The liner 40 comprises EAP portions 13, 21 on both sides of the device. The drug delivery layer 13 and EAP mechanical layer 21 are positioned on opposite sites of the liner, the “outside” respectively “inside” of the device. The device further comprises an insulating layer 24 to allow for individual control of the mechanical and drug delivery functions.

FIGS. 5a and 5b illustrate further alternative designs of a drug delivery liner. The liner 42 (FIG. 5a) comprises different drug delivery sections 13 and 14 that may contain different substances A and B and that may be individually controllably releasable. This allows for more complex delivery schemes. The substances A and B may be simultaneously released or sequentially, depending on the needed “procedure” for treatment of the disease in question. It is contemplated that A and B may be precursors or prodrugs to active substances A′ and B′ that are formed after release from the EAP sections by for instance by metabolization (e.g. hydrolysis) or enzymatic reactions of A and B. It is further contemplated that A and B are precursors and that when they are combined form substance C, the active drug. It is also contemplated that B may be a catalyst, initiator or enzyme that transforms the precursor A into the active substance A′.

It is contemplated that the liner may comprise more than two different drug delivery sections 13 and 14, allowing for more than two different substances to be released. FIG. 5b illustrates a liner 44 with yet another lay-out. In this case the drug delivery portions 13 are designed as “pads” or “islands” in a layer of EAP that has a mechanical function 21.

FIGS. 3-5 show different layouts of the liner. Combinations (e.g. pads of drug A in layer of drug B, i.e. combine FIG. 5a and FIG. 5b) or other variants of these are plausible.

The drug delivery scheme may be complex: the drug release may be pulsed according to a specific time pattern, the different substances may be released simultaneously, sequentially, or alternating, all dependent on the optimal treatment for the disease in question.

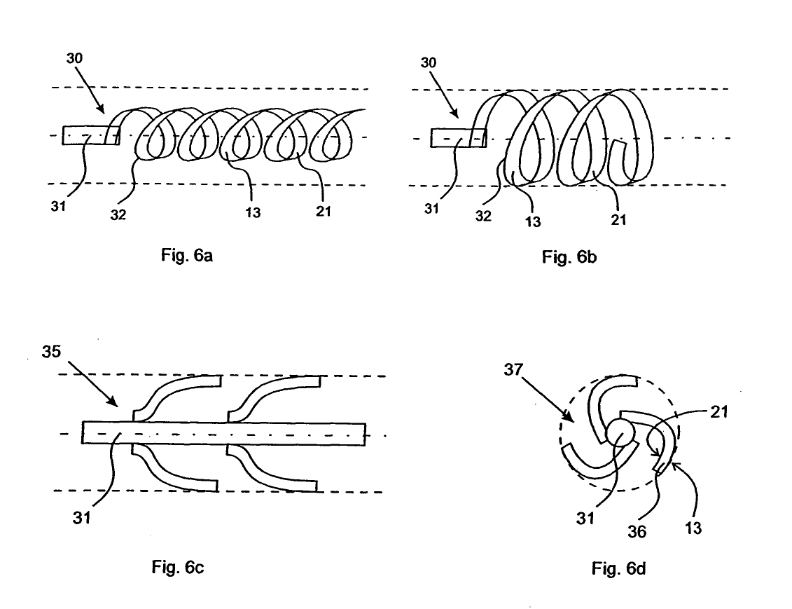

FIGS. 6a-6b shows another embodiment. This is similar to the above mentioned liner, however the drug delivery tool 30 is spiral shaped, see WO03039859, the entire contents of which is hereby incorporated by reference. A spiral 32 that comprises a drug delivery portion 13 and an EAP layer 21 is mounted on a part of medical device 31, e.g. a guide wire, or a catheter. The spiral may have a cross section as illustrated in FIG. 4. The rigid beams may be positioned at an oblique angle, which will generate a spiral motion as taught by WO03039859. When inserted into the body lumen, the spiral is in a contracted (FIG. 6a) or straight (not shown) shape. Arriving at the position of the treatment the device is first mechanically activated by actuating the mechanical EAP part 21. This will cause the spiral to press against the lumen wall with the drug delivery portion 13 facing the wall surface (FIG. 6b). Hereafter, the drug delivery portion is activated and the substances are delivered at the site of interest.

FIGS. 6c and 6d show further embodiments of the disclosure. FIG. 6c shows a device 35 that comprises at least one (four shown) “flap” or “wing” 36, which comprises a drug delivery portion 13 and an EAP portion 21, mounted on a part of the medical device 35, e.g. a guide wire, or a catheter. The flaps may, in the deactivated state (not shown), be substantially flat against the medical device 31, for easy insertion into the body. In the activated state (FIG. 6c) a part of the flap is pressed onto the lumen wall by an (electrically) activated means, such as an EAP portion 21, bringing the EAP drug delivery 13 portion into contact with the lumen wall. The flaps are arranged longitudinally to the central line of the medical device.

FIG. 6d shows a device 37 that is similar to the device 35 of FIG. 6c. The device 37 comprises at least one “flap” or “wing” 36 (three shown in FIG. 6d), however in this case the flaps are arranged perpendicular to the central line of the medical device. In the deactivated state, the flaps are laying flat onto (wholly or partly wound around) the device. In the activated state the flaps fold out and press against the lumen wall. It is possible to mount the flaps on the medical device at other angles than 0 or 90 degrees as is shown in FIGS. 6c and d. The flaps may be formed as shown in FIGS. 3a, 3b, 4, 5a, 5b, or other configurations as justified/implied/set forth by the design criteria.

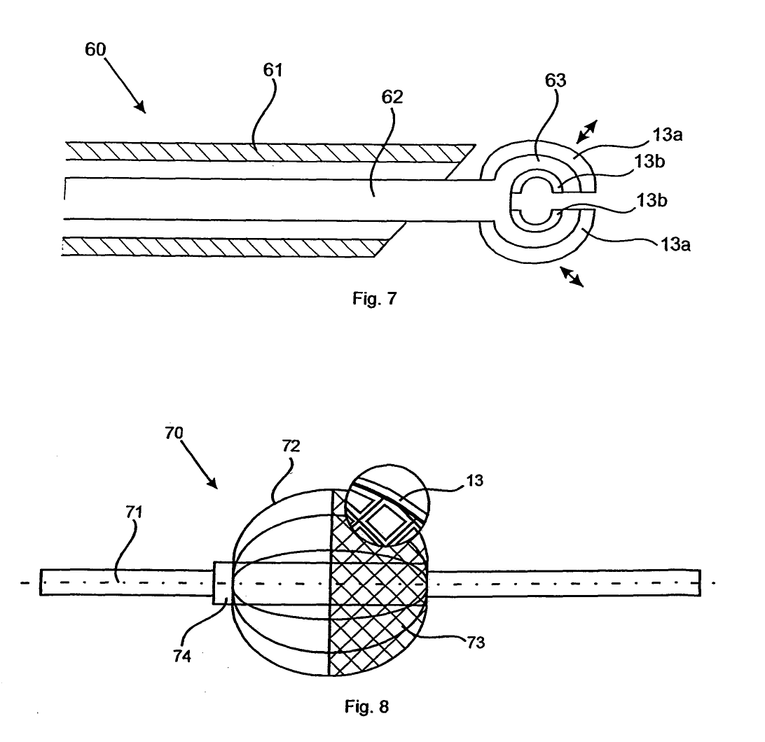

FIG. 7 shows yet another embodiment, wherein the device comprises a microsurgical tool 60 that may be inserted into the body through a cannula or catheter 61 for instance as disclosed in WO 00/78222. The tool 63 that may be a pair of clamps or forceps that are mounted on a needle like part 62 and comprises EAP drug delivery portions 13a, 13b. The portions may be positioned on the outside 13a of the forceps 63 and/or on the inside 13b of the forceps 63. This allows for the administration of the drug during the procedure in which the forceps are used.

FIG. 8 shows another embodiment, wherein the device is a filter apparatus, that is used in minimally invasive procedures, where the surgeon desires to capture particulates that are released during the procedure. Such devices are known to those skilled in the art.

One example is disclosed in US2004/00879821, the entire contents of which is hereby incorporated by reference. The device comprises a guide wire 71 over which a tube 74 slides that comprises a filter element 73 and actuation means 72. The filter assembly is introduced into the body in a contracted state (not shown). Once in place the practitioner deploys the filter by the actuation means 72. This means maybe a shape memory alloy, pull wires, electroactive polymers or any other suitable means as known by those skilled in the art or as used in the field. The filter element 73 may be a mesh or porous material that will filter particulate material (such as emboli from the blood) while permitting sufficient perfusion therethrough. The filter is partially or completely covered with a drug delivery portion 13 as shown in FIG. 8. The substances that may be released from the portion may e.g. be active so as to initiate dissolution of the emboli.

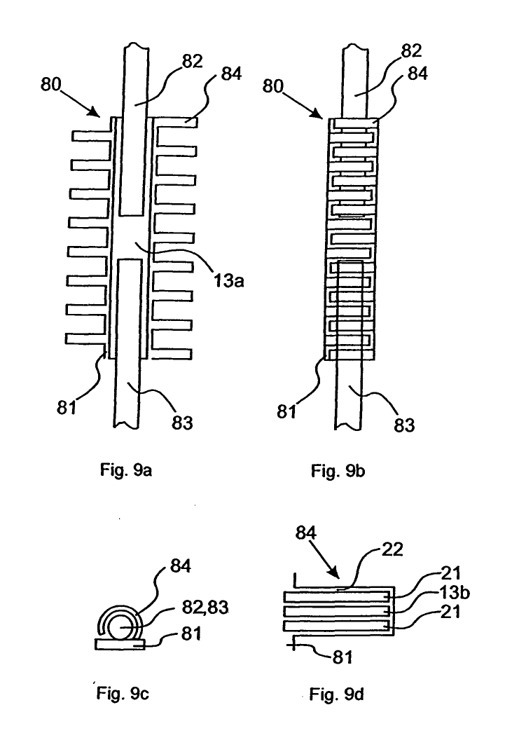

FIG. 9 illustrates a neural connector 80, such as the neural connector disclosed in WO 00/78222, where a number of EAP actuators or “fingers” 84 coil around a nerve to make a tight hold to the nerve. Two separate nerve endings 82 and 83 are joined with the help of a common neural connector 80. The two nerve endings could have been separated due to a trauma or cut, e.g. as a step in a surgical procedure. The neural connector further comprises drug delivery areas, 13a and/or 13b, to stimulate regrowth of the nerve and/or direct the growth of different neurons (e.g. motory and sensory neurons) by release of for instance growth factors.

FIG. 9a shows the neural connector 80 in an open state. The fingers or EAP actuators 84 (only one numbered) that may be mounted on a “base” 81 are opened. The connector 80 further comprises a drug delivery area 13a.

FIG. 9b shows the connector 80 in a closed state. The fingers 84 grab and hold the nerve endings 82 and 83, and the drug may be released at this point.

FIG. 9c shows a cross section of the device showing how a finger 84 coils around a nerve ending. The fingers 84 comprise at least an electroactive polymer 21 and a non-electroactive polymer layer 24, such as gold, and may even comprise a third, non-EAP layer. Possible cross sections of such an EAP actuator are shown in FIGS. 3b and 4. FIG. 9d illustrates a possible lateral layout of the fingers comprising the EAP areas 21 that exercise the mechanical function and a drug delivery area 13b.

The drug delivery area 13 may be only integrated on a part of the device, such as the “base” (illustrated as 13a in FIG. 9a) or only on the fingers (illustrated as 13b in FIG. 9b), or on both. It may be formed as one large area that covers a major part or the device area (as illustrated in FIG. 9a) or it may be segmented, patterned and/or comprise several different areas that comprise different types of drugs (see for instance FIGS. 5a and 5b). This may allow for the drugs to be released in a way as would be required for optimal stimulation of the nerve regrowth: The drugs may be released in a pulsed manner; or in a gradient generating manner; different kinds of drug may be released in sequence, on a specific time scale/pattern.

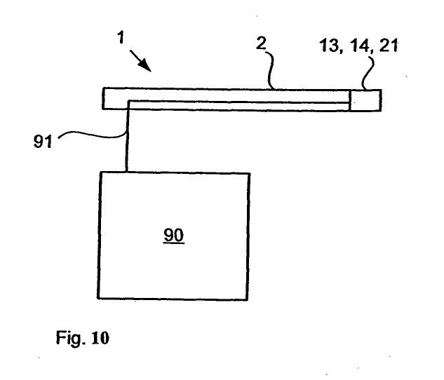

FIG. 10 schematically illustrates a system, comprising a medical device 1 having a body insertable part 2 and a control device 90. The control device may be connected to the EAP portions 13, 14 and to the actuator 21 by wires 91.

The skilled person understands that this is merely a demonstration of the principle and that the scope of the appended claims is not limited to these examples. The skilled person also realizes that other applications can be plausible as, for example, a liner for release of chemical substances inside microsystems.

Further non-limiting examples of substances that may be released include anti inflammatory substances, such as Dexamethasone Phosphate and salicylic acid; anti-spasm/thrombosis substances, such as Alprostadil® (Prostaglandin E-1) and Lidocain®; Anti-arrytmi and anti-inflammatory substances, such as adenosine; anti-coagulants, such as Heparin®, Clopidrogel®, bisulfate and Urokinase®; antioxidants, such as Probucol® and Retinoic acid; antiplatelet drugs, such as Trapidil® (triazolopyrimidine); anti-proliferative substances, such as Angiopeptin (V)®, Methotrexate®, Mitomycine®, 2-chloro-deoxyadenosine, actinomycin-D, C-myc antisense, Vincristine® and sodium nitroprusside; anti-sense substances, such as Resten NG®; tranilast, antibiotic substances, such as Cromolyn sodium salt; cytokine substances, involved in processes essential to the growth, such as VEGF; cytotoxic antibiotics (anti-cancer drug), such as doxorubicin and mytomycin; vascular remodeling substances, such as Cytochalasin B®; estrogen, such as 17β-estradiol(oestrodiol); immunosuppresants, such as Tranilast®, mycophenolic acid, Tacrolimus® (FK 506), Pimecrolimus®, Zotarolimus® ABT-578; Leflunomide®, Mizoribine®, (methyl)prednisolone, Sirolimus® (rapamycin), Cyclosporine®, Clodronate®; mettaloproteinase inhibitors, such as Batimastat® and Marimastat®; neurotransmitters, such as dopamine, D-aspartic acid, Tryptophane® (metabolizes to serotonin), GABA (Gamma-aminobutyric acid), ACh (AcetylCholine), norepinephrine (Noradrenalin); non steroidal anti-inflammatory substances, such as Naproxen®, Profener® (2-Arylpropionic acids), ibuprofen etc., arylalkanoic acids(diclofenac etc); pain killers, such as paracetamol; platelet glycoprotein lIlb/illa inhibitors, such as abciximab; prodrugs, such as cortisol-21-phosphate; synthetic angiopeptins, such as Somatostatin®; synthetic prostacyclin, such as Iloprost®; Tumour supressors, such as Halofuginone®; and vasodilators, such as Papaverine®, Epinephrine® (Adrenaline), Prostacyclin®, Theobromine®, Forskoline®.

Other non-limiting examples of substances include L-arginin, Linsidomine®, Limulin®, Pegylated hirudin, Propyl hydroxylase, ATP, Corticosterone®, Albumine®, Rosiglitazone®.

1. A medical device for introduction into a body, comprising:
a body-insertable part having a first electroactive polymer, integrated with the body-insertable part and comprising an electrically controllably releasable first substance.

2-44. (canceled)
